(19) United States
(12) Patent Application Publication
(10) Pub. No.: US 2001/0051612 A1
(43) Pub. Date: Dec. 13, 2001

(54) 2-Thioether A2A receptor agonists

(76) Inventor: Gloria Cristalli, Camerino (IT)

Correspondence Address: A. Blair Hughes, McDonnell Boehnen Hulbert & Berghoff, 32nd Floor, 300 S. Wacker Drive, Chicago, IL, 60606, US

(21) Appl. No.: 09/733,196

(22) Filed: Dec. 8, 2000

Related U.S. Application Data
(60) Provisional application No. 60/184,475, filed on Feb. 23, 2000.

(51) Int. Cl. A61K 31/7076; C07H 19/22

(52) U.S. Cl. 514/45; 536/27.13

(57) ABSTRACT

2-adenosine propargyl phenyl ether compositions having the following formula:

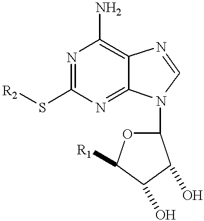

and methods for using the compositions as A_{2A} receptor agonists to stimulate mammalian coronary vasodilatation for therapeutic purposes and for purposes of imaging the heart.

### This application claims priority to U.S. Provisional Patent Application Ser. No. 60/184,475 filed on Feb. 23, 2000

### BACKGROUND OF THE INVENTION

(1) Field of Invention

This invention includes 2-adenosine thioether compositions that are useful as A_{2A} receptor agonists. The compositions of this invention are vasodialating agents that are useful in a heart imaging to identify mammals, and especially humans who are suffering from coronary disorders such as poor coronary perfusion which is indicative of coronary artery disease (CAD). The compositions of this invention can also be used as therapeutics for coronary artery disease.

(2) Description of the Art

Pharmacological stress is frequently induced with non-specific adenosine agonists such as adenosine or dipyridamole in patients with suspected CAD before imaging with T1 scintigraphy or echocardiography. Both drugs effect dilation of the coronary resistance vessels by activation of cell surface A₂ receptors. Although pharmacological stress was originally introduced as a mean of provoking coronary dilation in patients unable to exercise, several studies have shown that the prognostic value of ²⁰¹T1 or echocardiographic imaging in patients subjected to pharmacological stress with adenosine or dipyridamole was equivalent to patients subjected to traditional exercise stress tests. However, there is a high incidence of drug-related adverse side effects during pharmacological stress imaging with these drugs such as headache and nausea, that could be improved with new therapeutic agents.

Adenosine A_{2B} and A₃ receptors are involved in a mast cell degranulation and, therefore, asthmatics are not given non-specific adenosine agonists to induce a pharmacological stress test. Additionally, adenosine stimulation of the A₁ receptor in the atrium and A-V mode will diminish the S-H interval which can induce AV block. (N. C. Gupto et al.; J. Am Coll. Cardiol; (1992) 19: 248-257). Also, stimulation of the adenosine A1 receptor by adenosine may be responsible for the nausea since the A₁ receptor is found in the intestinal tract. (J. Nicholls et al.; Eur. J. Pharm.(1997) 338 (2) 143-150).

Animal data suggests that specific adenosine A_{2A} subtype receptors on coronary resistance vessels mediate the coronary dilatory responses to adenosine, whereas subtype A_{2B} receptor stimulation relaxes peripheral vessels (note: the latter lowers systemic blood pressure). As a result there is a need for pharmaceutical compositions that are selective A_{2A} receptor agonists that have no pharmacological effect as a result of stimulating the A₁ receptor in vivo. Furthermore, there is a need for A_{2A} receptor agonists that have a short half-life, and that are well tolerated by patients undergoing pharmacological coronary stress evaluations.

### SUMMARY OF THE INVENTION

In one aspect, this invention includes 2-adenosine thioether compositions that are useful A_{2A} receptor agonists.

In another aspect, this invention includes pharmaceutical compositions including 2-adenosine thioether compounds that are well tolerated with few side effects.

Still another aspect of this invention are 2-adenosine thioether compositions that can be easily used in conjunction with radioactive imaging agents to facilitate coronary imaging.

In one embodiment, this invention includes 2-adenosine thioether compositions having the following formula:

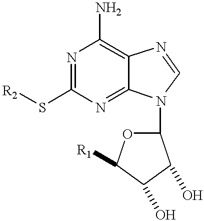

In another embodiment, this invention includes methods for using compositions of this invention to stimulate coronary vasodilatation in mammals, and especially in humans, for stressing the heart induced steal situation for purposes of imaging the heart.

In still another embodiment, this invention is pharmaceutical compositions of matter comprising one or more compositions of this invention and one or more pharmaceutical excipients.

### DESCRIPTION OF THE CURRENT EMBODIMENT

The compositions of this invention include a class of thioether substituted 2-adenosine compounds having the following formula:

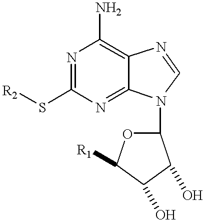

R¹is —CH₂OH, and —C(═O)NR⁷R⁸;

R² is individually selected from the group consisting of C₁₋₁₅ alkyl, aryl, and heteroaryl, which alkyl, aryl, and heteroaryl are optionally substituted with from 1 to 2 substituents independently selected from the group consisting of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, and wherein each optional heteroaryl, aryl, and heterocyclyl substitution substituent is further optionally substituted with halo, alkyl, CF₃, amino, mono- or di-alkylamino, SR²⁰, S(O)R²², SO₂R²², SO₂N(R²⁰)₂, CN, or OR²⁰;

R⁷ and R⁸ are each independently selected from H, and C₁₋₆ alkyl optionally substituted with a substituent independently selected from the group consisting of aryl and heteroaryl.

R²⁰ is selected from the group consisting of H, C₁₋₆ alkyl, aryl, and heteroaryl, which alkyl, aryl, and heteroaryl are each optionally substituted with from 1 to 2 substituents independently selected from halo, alkyl, mono- or dialkylamino, alkyl or aryl or heteroaryl amide, CN, O—C₁₋₆ alkyl, CF₃,; and

R²² is selected from the group consisting of C₁₋₆ alkyl, aryl, and heteroaryl which alkyl, aryl, and heteroaryl are each optionally substituted with from 1 to 2 substituents independently selected from halo, alkyl, mono- or dialkylamino, CN, —O—C₁₋₆ alkyl, and CF₃.

Preferably, R¹ is CH₂OH or C(O)NHEt; R² is selected from the group consisting of C₁₋₆ alkyl and aryl which alkyl is optionally substituted with 1 substituent independently selected from the group consisting of aryl, OR²⁰, COR²⁰, CO₂R²⁰, CON(R²⁰)₂ which aryl is further optionally substituted by halo OR²⁰, COR²⁰, CO₂R²⁰, CON(R²⁰)₂; and

R20 is selected from the group consisting of H, and C₁₋₂ alkyl.

More preferably R¹ is CH₂OH or C(O)NHEt; R² is selected from the group consisting of C₁₋₆ alkyl and aryl which alkyl is optionally substituted with 1 substituent independently selected from the group consisting of aryl and OR²⁰ and which aryl is further optionally substituted by halo; and R²⁰ is H.

In a further preferred embodiment preferably R¹ is CH₂OH or C(O)NHEt; R² is selected from the group consisting of is individually selected from the group consisting of C₁₋₆ alkyl and aryl which alkyl is optionally substituted with 1 substituent independently selected from the group consisting of aryl and OR²⁰; and R²⁰ is H.

In still a further preferred embodiment, the compounds of this invention are selected from the group consisting of (4S, 2R, 3R, 5R)-2-(6-amino-2-phenylthiopurin-9-yl)-5-(hydroxymethyl)oxolane-3,4-diol, (4S, 2R, 3R, 5R)-2-(6-amino-2-phenylmethylthiopurin-9-yl)-5-(hydroxymethyl)oxolane-3,4-diol, (4S, 2R, 3R, 5R)-2-(6-amino-2-phenylethylthiopurin 9-yl)-5-(hydroxymethyl)oxolane-3,4-diol, (4S, 2R, 3R, 5R)-2-(6-amino-2-phenylpropylthiopurin-9-yl)-5-(hydroxymethyl)oxolane-3,4-diol, (4S, 2R, 3R, 5R)-2-(6-amino-2-pentylthiopurin-9-yl)-5-(hydroxymethyl)oxolane-3,4-diol, (4S, 2R, 3R, 5R)-2-(6-amino-2-(4-hydroxybutylthio)purin-9-yl)-5-(hydroxymethyl)oxolane-3,4-diol, (4S, 2R, 3R, 5R)-2-(6-amino-2-cyclopentylthiopurin-9-yl)-5-(hydroxymethyl)oxolane-3,4-diol, [(2S, 3S 4R, 5R)-5-(6-amino-2-phenylthiopurin-9-yl)-3,4-dihydroxyoxolan-2yl]-N-ethylcarboxamide, [(2S, 3S, 4R, 5R)-5-(6-amino-2-(phenylmethylthio)purin-9-yl)-3,4-dihydroxyoxolan-2yl]-N -ethylcarboxamide, [(2S, 3S, 4R, 5R)-5-(6-amino-2-(phenylethylthio)purin-9-yl)-3,4-dihydroxyoxolan-2-yl]-N-ethylcarboxamide, [(2S, 3S, 4R, 5R)-5-(6-amino-2-(phenylpropylthio)purin-9-yl)-3,4-dihydroxyoxolan-2-yl]-N-ethylcarboxamide, [(2S, 3S, 4R, 5R)-5-(6-amino-2-pentylthiopurin-9-yl)-3,4-dihydroxyoxolan-2-yl]-N-ethylcarboxamide,a and mixtures thereof.

The following definitions apply to terms as used herein.

“Halo” or “Halogen”—alone or in combination means all halogens, that is, chloro (Cl), fluoro (F), bromo (Br), iodo (I).

“Hydroxyl” refers to the group —OH.

“Thiol” or “mercapto” refers to the group —SH. “Alkyl”—alone or in combination means an alkane-derived radical containing from 1 to 20, preferably 1 to 15, carbon atoms (unless specifically defined). It is a straight chain alkyl, branched alkyl or cycloalkyl. Preferably, straight or branched alkyl groups containing from 1- 15, more preferably 1 to 8, even more preferably 1-6, yet more preferably 1-4 and most preferably 1-2, carbon atoms, such as methyl, ethyl, propyl, isopropyl, butyl, t-butyl and the like. The term “lower alkyl” is used herein to describe the straight chain alkyl groups described immediately above. Preferably, cycloalkyl groups are monocyclic, bicyclic or tricyclic ring systems of 3-8, more preferably 3-6, ring members per ring, such as cyclopropyl, cyclopentyl, cyclohexyl, adamantyl and the like. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl. A substituted alkyl is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, amino sulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamnino, heteroarylcarbonylamino, or the like.

“Alkenyl”—alone or in combination means a straight, branched, or cyclic hydrocarbon containing 2-20, preferably 2-17, more preferably 2-10, even more preferably 2-8, most preferably 2-4, carbon atoms and at least one, preferably 1-3, more preferably 1-2, most preferably one, carbon to carbon double bond. In the case of a cycloalkyl group, conjugation of more than one carbon to carbon double bond is not such as to confer aromaticity to the ring. Carbon to carbon double bonds may be either contained within a cycloalkyl portion, with the exception of cyclopropyl, or within a straight chain or branched portion. Examples of alkenyl groups include ethenyl, propenyl, isopropenyl, butenyl, cyclohexenyl, cyclohexenylalkyl and the like. A substituted alkenyl is the straight chain alkenyl, branched alkenyl or cycloalkenyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, amino sulfonyl optionally N-mono- or N,N-di- substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, carboxy, alkoxycarbonyl, aryloxycarbonyl, heteroaryloxycarbonyl, or the like attached at any available point to produce a stable compound.

“Alkynyl”—alone or in combination means a straight or branched hydrocarbon containing 2-20, preferably 2-17, more preferably 2-10, even more preferably 2-8, most preferably 2-4, carbon atoms containing at least one, preferably one, carbon to carbon triple bond. Examples of alkynyl groups include ethynyl, propynyl, butynyl and the like. A substituted alkynyl refers to the straight chain alkynyl or branched alkenyl defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like attached at any available point to produce a stable compound.

“Alkyl alkenyl” refers to a group —R—CR′═CR′″ R″″, where R is lower alkyl, or substituted lower alkyl, R′, R′″, R″″ may independently be hydrogen, halogen, lower alkyl, substituted lower alkyl, acyl, aryl, substituted aryl, hetaryl, or substituted hetaryl as defined below.

“Alkyl alkynyl” refers to a groups —RC≡CR′ where R is lower alkyl or substituted lower alkyl, R′ is hydrogen, lower alkyl, substituted lower alkyl, acyl, aryl, substituted aryl, hetaryl, or substituted hetaryl as defined below.

“Alkoxy” denotes the group —OR, where R is lower alkyl, substituted lower alkyl, acyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

“Alkylthio” denotes the group —SR, —S(O)_{n═1-2}—R, where R is lower alkyl, substituted lower alkyl, aryl, substituted aryl, aralkyl or substituted aralkyl as defined herein.

“Acyl” denotes groups —C(O)R, where R is hydrogen, lower alkyl substituted lower alkyl, aryl, substituted aryl and the like as defined herein.

“Aryloxy” denotes groups —OAr, where Ar is an aryl, substituted aryl, heteroaryl, or substituted heteroaryl group as defined herein.

“Amino” denotes the group NRR′, where R and R′ may independently by hydrogen, lower alkyl, substituted lower alkyl, aryl, substituted aryl, hetaryl, or substituted hetaryl as defined herein or acyl.

“Amido” denotes the group —C(O)NRR′, where R and R′ may independently by hydrogen, lower alkyl, substituted lower alkyl, aryl, substituted aryl, hetaryl, substituted hetaryl as defined herein.

“Carboxyl” denotes the group —C(O)OR, where R is hydrogen, lower alkyl, substituted lower alkyl, aryl, substituted aryl, hetaryl, and substituted hetaryl as defined herein.

“Aryl”—alone or in combination means phenyl or naphthyl optionally carbocyclic fused with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Substituted aryl” refers to aryl optionally substituted with one or more functional groups, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heterocycle” refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl or benzo[b] thienyl) and having at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heteroaryl”—alone or in combination means a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group O, S, and N, and optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, amino sulfonyl optionally N-mono- or N,N-di- substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. Examples of heteroaryl groups are pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl and the like. A substituted heteroaryl contains a substituent attached at an available carbon or nitrogen to produce a stable compound. “Heterocyclyl”—alone or in combination means a non-aromatic cycloalkyl group having from 5 to 10 atoms in which from 1 to 3 carbon atoms in the ring are replaced by heteroatoms of O, S or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. Examples of heterocyclyl groups are tetrahydrofuranyl, dihydropyridinyl, piperidinyl, pyrrolidinyl, piperazinyl, dihydrobenzofuryl, dihydroindolyl, and the like. A substituted hetercyclyl contains a substituent nitrogen attached at an available carbon or nitrogen to produce a stable compound. “Substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Aralkyl” refers to the group —R—Ar where Ar is an aryl group and R is lower alkyl or substituted lower alkyl group. Aryl groups can optionally be unsubstituted or substituted with, e.g., halogen, lower alkyl, alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Heteroalkyl” refers to the group —R—Het where Het is a heterocycle group and R is a lower alkyl group. Heteroalkyl groups can optionally be unsubstituted or substituted with e.g., halogen, lower alkyl, lower alkoxy, alkylthio, acetylene, amino, amido, carboxyl, aryl, aryloxy, heterocycle, substituted heterocycle, hetaryl, substituted hetaryl, nitro, cyano, thiol, sulfamido and the like.

“Heteroarylalkyl” refers to the group —R—HetAr where HetAr is an heteroaryl group and R lower alkyl or substituted lower alkyl. Heteroarylalkyl groups can optionally be unsubstituted or substituted with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like. “Cycloalkyl” refers to a divalent cyclic or polycyclic alkyl group containing 3 to 15 carbon atoms.

“Substituted cycloalkyl” refers to a cycloalkyl group comprising one or more substituents with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloheteroalkyl” refers to a cycloalkyl group wherein one or more of the ring carbon atoms is replaced with a heteroatom (e.g., N, O, S or P).

“Substituted cycloheteroalkyl” refers to a cycloheteroalkyl group as herein defined which contains one or more substituents, such as halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloalkyl” denotes the group —R-cycloalkyl where cycloalkyl is a cycloalkyl group and R is a lower alkyl or substituted lower alkyl. Cycloalkyl groups can optionally be unsubstituted or substituted with e.g. halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloheteroalkyl” denotes the group —R-cycloheteroalkyl where R is a lower alkyl or substituted lower alkyl. Cycloheteroalkyl groups can optionally be unsubstituted or substituted with e.g. halogen, lower alkyl, lower alkoxy, alkylthio, amino, amido, carboxyl, acetylene, hydroxyl, aryl, aryloxy, heterocycle, substituted heterocycle, hetaryl, substituted hetaryl, nitro, cyano, thiol, sulfamido and the like.

The compounds of this invention can be prepared as outlined in the general Schemes 1-2 below. Compounds with general formula II can be prepared as shown in Scheme 1. The 2-iodoadenosine can be prepared from guanosine using a diazonium mediated radical process as described by Nair and Richardson ( Synthesis (1982) 670-672). The displacement of the 2-iodo group can be accomplished by warming the appropriately substituted mercaptan in a dimethylformamide solution in the presence of base (eg. potassium carbonate). Compounds 1-7 were prepared using the method described in Scheme 1.

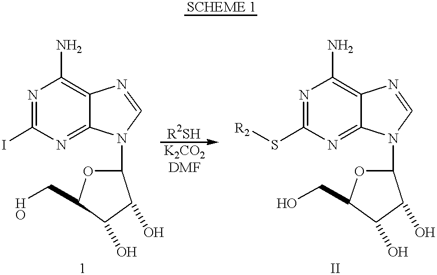

The yields, reaction time, chromatography solvents, and melting points are recorded in Table 1.

TABLE 1
Preparation of Compounds Reported in Scheme 1

| Compd | | | | yield | |
|---|---|---|---|---|---|
| no. | R | time (h) | chromatography solvent | % | mp (° C.) |
| 1 | C₆H₅ | 20 | CHCl₃—CH₃OH | 40 | 137-139 |
| | | | (88:12) | | lit^{a} 125- |
| | | | | | 126 |
| 2 | CH₂C₆H₅ | 20 | CHCl₃—CH₃OH—cC₆H₁₂ | 63 | 158 dec |
| | | | (75:15:10) | | lit^{a} 158 |
| 3 | CH₂CH₂C₆H₅ | 16 | CHCl₃—CH₃OH—cC₆H₁₂ | 50 | 205 dec |
| | | | (77:13:10) | | |
| 4 | CH₂CH₂CH₂C₆H₅ | 16 | CHCl₃—CH₃OH—cC₆H₁₂ | 37 | 92-95 |
| | | | (78:12:10) | | |
| 5 | CH₂CH₂CH₂CH₂CH₃ | 20 | CHCl₃—CH₃OH—cC₆H₁₂ | 32 | 177 dec |
| | | | (76:14:10) | | lit^{b} 148- |
| | | | | | 151 |
| 6 | CH₂CH₂CH₂CH₂OH | 16 | CHCl₃—CH₃OH—cC₆H₁₂ | 34 | 88-90 |
| | | | (72:18:10) | | |
| 7 | H₉ | 20 | CHCl₃—CH₃OH—cC₆H₁₂ | 38 | 213 dec |
| | | | (80:14:6) | | lit^{b} 223- |
| | | | | | 225 |

Compounds with general formula V can be prepared as shown in Scheme 2. Compound 2, which can be obtained by reacting compound 1 with 2,2-dimethoxypropane in the presence of an acid, can be oxidized to the carboxylic acid 3, based on structurally similar compounds, using potassium permanganate or pyridinium chlorochromate, or TEMPO etc., (M. Hudlicky, (1990) Oxidations in Organic Chemistry, ACS Monographs, American Chemical Society, Washington D.C.; B. Cox et al, WO 9828319) to compound 3. Reaction of a primary or secondary amine with the formula HNR⁶R⁷, and compound 3 using DCC (M. Fujino et al., Chem. Pharm. Bull. (1974), 22, 1857), PyBOP (J. Martinez et al., J. Med. Chem. (1988) 28, 1874) or PyBrop (J. Caste et al. Tetrahedron, (1991), 32, 1967) coupling conditions can afford compound V. Alternatively, when NR⁷R⁸ corresponds to a simple alkyl amine (eg ethylamine), then the method utilizing an acid chloride mediated ester formation can be used as described previously by Cristalli et al (J. Med. Chem. 1992, 35, p 2363-2368). Deprotection of compound III can be performed by heating with 80% aqueous acetic acid (T. W. Greene and P. G. M. Wuts, (1991) Protective Groups in Organic Synthesis, A. Wiley-Interscience publication) or with anhydrous hydrochloric acid (4N) to obtain compound IV. Compound V can be obtained by warming the 2-iodo derivative in the presence of the appropriately substituted mercaptan in the presence of a base (eg. potassium carbonate). Compounds 1a through 5a were prepared in the manner of scheme 2 as described above, and the yields, chromatography conditions, and melting points for the final step are listed in Table 2 .

TABLE 2

| Compd | | | | yield | mp |
|---|---|---|---|---|---|
| no. | R | time (h) | chromatography solvent | % | (° C.) |
| 1a | C₆H₅ | 6 | CHCl₃—CH₃OH—cC₆H₁₂ | 46 | 122- |
| | | | (80:10:10) | | 125 |
| 2a | CH₂C₆H₅ | 6 | CHCl₃—CH₃OH—cC₆H₁₂ | 78 | 245 |
| | | | (80:10:10) | | dec |
| 3a | CH₂CH₂C₆H₅ | 6 | CHCl₃—CH₃OH—cC₆H₁₂ | 57 | 97-99 |
| | | | (80:10:10) | | |
| 4a | CH₂CH₂CH₂C₆H₅ | 16 | CHCl₃—CH₃OH | 31 | 185- |
| | | | (90:10) | | 188 |
| 5a | CH₂CH₂CH₂CH₂CH₃ | 36 | CHCl₃—CH₃OH—cC₆H₁₂ | 11 | >270 |
| | | | (80:10:10) | | dec |

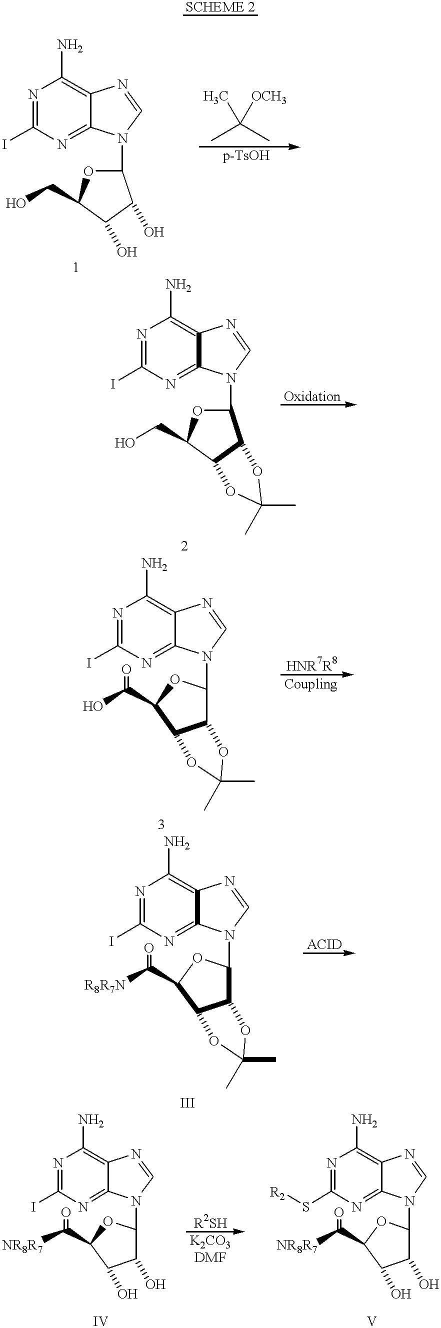

The methods that may be used to prepare the compounds of this invention are not limited to those described above. Additional methods can be found in the following sources and are included herein by reference (J. March, Advanced Organic Chemistry; Reaction Mechanisms and Studies (1992), A Wiley Interscience Publications).

Compounds of this invention are useful in conjunction with radioactive imaging agents to image coronary activity. The compounds of this invention are A_{2A} agonists that provide specific activation of adenosine A_{2A} receptors in the coronary vessels as opposed to adenosine A₁ receptors in the atrium and AV-node and/or A_{2B} receptors in peripheral vessels, thus avoiding undesirable side-effects. Upon administration in a therapeutic amount, the compositions of this invention cause coronary blood vessels to vasodilate to induce coronary steal wherein healthy coronary vessels steal blood from unhealthy vessels resulting in lack of blood flow to heart tissues. Coronary imaging then identifies coronary regions with healthy and unhealthy blood flow. Lower doses of the A_{2A} agonists may provide beneficial coronary vasodilatation (less severe) in the treatment of chronic CAD.

As A_{2A} agonists, the compositions of this invention are also useful in adjunctive therapy with angioplasty to induce dilation, inhibit platelet aggregation, and as a general anti-inflammatory agent. A_{2A} agonists, such as the compositions of this invention, can provide the therapeutic benefits described above by preventing neutrophil activation (Purinergic Approaches in Experimental Therapeutics K. A. Jacobson and M. F. Jarvis 1997 Wiley, New York). The compounds of this invention are also effective against a condition called no-reflow in which platelets and neutrophils aggregate and block a vessel. As A_{2A} agonists, the compositions of this invention are effective against no-reflow by preventing neutrophil and platelet activation (e.g., they are believed to prevent release of superoxide from neutrophils). As A_{2A} agonists, the compositions of this invention are also useful as cardioprotective agents through their anti-inflammatory action on neutrophils. Thus, in situations when the heart will go through an ischemic state such as a transplant, they will be useful.

This invention also includes pro-drugs of the above-identified A_{2A} agonists. A pro-drug is a drug which has been chemically modified and may be biological inactive at its site of action, but which will be degraded or modified by one or more enzymatic or in vivo processes to the bioactive form. The pro-drugs of this invention should have a different pharmacokinetic profile to the parent enabling improved absorption across the mucosal epithelium, better salt formulation and/or solubility and improved systemic stability. The above-identified compounds may be preferably modified at one or more of the hydroxyl groups. The modifications may be (1) ester or carbamate derivatives which may be cleaved by esterases or lipases, for example; (2) peptides which may be recognized by specific or non-specific proteinase; or (3) derivatives that accumulate at a site of action through membrane selection or a pro-drug form or modified pro-drug form, or any combination of (1) to (3) above.

The compositions of this invention may be administered orally, intravenously, bolus, through the epidermis or by any other means known in the art for administering a therapeutic agents. The method of treatment comprises the administration of an effective quantity of the chosen compound, preferably dispersed in a pharmaceutical carrier. Dosage units of the active ingredient are generally selected from the range of 0.01 to 100 mg/kg, but will be readily determined by one skilled in the art depending upon the route of administration, age and condition of the patient. This dose is typically administered in a solution about 5 minutes to about an hour or more prior to coronary imaging. No unacceptable toxicological effects are expected when compounds of the invention are administered in therapeutic amounts.

If the final compound of this invention contains a basic group, an acid addition salt may be prepared. Acid addition salts of the compounds are prepared in a standard manner in a suitable solvent from the parent compound and an excess of acid, such as hydrochloric, hydrobromic, sulfuric, phosphoric, acetic, maleic, succinic, or methanesulfonic. The hydrochloric salt form is especially useful. If the final compound contains an acidic group, cationic salts may be prepared. Typically the parent compound is treated with an excess of an alkaline reagent, such as hydroxide, carbonate or alkoxide, containing the appropriate cation. Cations such as Na⁺, K⁺, Ca⁺² and NH₄⁺ are examples of cations present in pharmaceutically acceptable salts. Certain of the compounds form inner salts or zwitterions which may also be acceptable.

Pharmaceutical compositions including the compounds of this invention, and/or derivatives thereof, may be formulated as solutions or lyophilized powders for parenteral administration. Powders may be reconstituted by addition of a suitable diluent or other pharmaceutically acceptable carrier prior to use. If used in liquid form the compositions of this invention are preferably incorporated into a buffered, isotonic, aqueous solution. Examples of suitable diluents are normal isotonic saline solution, standard 5% dextrose in water and buffered sodium or ammonium acetate solution. Such liquid formulations are suitable for parenteral administration, but may also be used for oral administration. It may be desirable to add excipients such as polyvinylpyrrolidinone, gelatin, hydroxycellulose, acacia, polyethylene glycol, mannitol, sodium chloride, sodium citrate or any other excipient known to one of skill in the art to pharmaceutical compositions including compounds of this invention.

Alternatively, the pharmaceutical compounds may be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, teffa alba, magnesium stearate or stearic acid, talc, pectin, acacia, agar or gelatin. The carrier may also include a sustained release material such as glycerol monostearate or glycerol distearate, alone or with a wax. The amount of solid carrier varies but, preferably, will be between about 20 mg to about 1 gram per dosage unit. The pharmaceutical dosages are made using conventional techniques such as milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly or filled into a soft gelatin capsule. It is preferred that the compositions of this invention are administered as a solution either orally or intravenously.

The Examples which follow serve to illustrate this invention. The Examples are intended to in no way limit the scope of this invention, but are provided to show how to make and use the compounds of this invention. In the Examples, all temperatures are in degrees Centigrade.

### EXAMPLE 1

### Preparation of 2-(ar)alkylthioadenosines (1-7)

A mixture of 2-iodoadenosine (1, 0.2 g, 0.51 mmol)′ prepared as described in V. Nair Synthesis (1982) 670-672) in 5 mL of dry DMF, 2.55 mmol of the appropriate mercaptan, and solid K₂CO₃ (150 mg, 1.05 mmol) was heated in a steel bomb at 120° C. for the time reported in Table 1. The reaction mixture was concentrated in vacuo and the residue was chromatographed on a silica gel column eluting with the suitable mixture of solvents (Table 1) to give 1-7 as chromatographically pure solids.

### (4S, 2R, 3R, 5R)-2-(6-amino-2-phenylthiopurin-9-yl)-5-(hydroxymethyl)oxolane-3,4-diol, (1)

¹H NMR (Me₂SO-d₆) δ 3.32 (m, 2H, CH₂-5′), 3.84 (m, 2H, H-4′ and H-3′), 4.55 (m, 1H, H -2′), 5.67 (d, J=6.1 Hz, 1H, H-1′), 7.45 (m, 5H, H-Ph and NH₂), 7.60 (m, 2H, H-Ph), 8.24 (s, 1H, H-8).

### (4S, 2R. 3R, 5R)-2-(6-amino-2-phenylmethylthiopurin-9-yl)-5-(hydroxymethyl)-oxolane -3,4-diol(2)

¹H NMR (Me₂SO-d₆) δ3.60 (m, 2H, CH₂-5′), 3.93 (m, 1H, H-4′), 4.15 (m, 1H, H-3′), 438 (s, 2H, CH₂S), 4.57 (m, 1H, H-2′), 5.88 (d, 1H, J=6.1 Hz, H-1′), 7.30 (m, 3H, H-Ph), 7.46 (m 4H, H-Ph and NH₂), 8.27 (s, 1H, H-8).

### (4S, 2R, 3R, 5R)-2-(6-amino-2-phenylethylthiopurin-9-yl)-5-(hydroxymethyl)oxolane -3,4-diol (3 )

¹H NMR (Me₂SO-d₆) δ 2.99 (m, 2H, CH₂Ph), 3.32 (m, 2H, CH₂S), 3.61 (m, 2H, CH₂-5′) 3.96 (m, 1H, H-4′), 4.15 (m, 1H, H-3′), 4.62 (t, 1H, J=5.5 Hz, H-2′), 5.92 (d, 1H, J=6.1 Hz, H-1′), 7.30 (m, 5H, H-Ph), 7.45 (bs, 2H, NH₂), 8.29 (s, 1H, H-8).

### (4S, 2R, 3R, 5R)-2-(6-amino-2-phenylethylthiopurin-9-yl)-5-(hydroxymethyl)oxolane -3,4-diol (4)

¹H NMR (Me₂SO-d₆) δ 1.98 (m, 2H, CH₂CH₂Ph), 2.74 (t, J=7.1 Hz, 2H, CH₂Ph), 3.09 (m, 2H, CH₂S), 3.94 (m, 1H, H-4′), 4.16 (m, 1H, H-3′), 4.63 (m, 1H, H-2′), 5.84 (d, J=5.9 Hz, 1H, H-1′), 7.25 (m, 5H, H-Ph), 7.39 (bs, 2H, NH₂), 8.25 (s, 1H, H-8).

### (4S, 2R,3R, 5R)-2-(6-amino-2-pentyltiopurin-9-yl)-5-(hydroxymethymethyl)oxolane-3,4-diol (5)

¹H NMR (Me₂SO-d₆) δ 0.89 (t, 3H, J=6.6 Hz, CH₂CH₃), 1.35 (m, 4H, (CH₂)₂CH₃), 165 (m 2H, CH₂CH₂S), 304 (m, 2H,CH₂S), 3.60 (m, 2H-CH₂5′) 3.92 (m, 1H, H-4′), 413 (m, 1H, H-3′), 4.62 (m, 1H, H-2′), 5.82 (d, 1H, J=5.1 Hz, H-1′), 7.38 (bs, 2H, NH₂), 8.24 (s, 1H, H -8).Anal. (C₁₅H₂₃N₅O₄S)

### (4S, 2R, 3R, 5R)-2-(6-amino-2-(4-hydroxybutylthio)purin-9-yl)-5-(hydroxymethyl)oxolane-3,4-diol (6)

¹H NMR (Me₂SO-d₆) δ1.66 (m, 4H, (CH₂)₂CH₂S), 3.11 (m, 2H, CH₂S), 3.54 (m, 2H, CH₂-5′and CH₂OH), 3.92 (m, 1H, H-4′), 4.15 (m 1H, H-3′), 4.44 (m, 1H, CH₂OH), 4.62 (m, 1H, H -2′), 5.83 (d, J=5.1 Hz, 1H, H-1′), 7.37 (bs, 2H, NH₂), 8.24 (s, 1H, H-8).

### (4S, 2R, 3R, 5R)-2-(6-amino-2-cyclopentylthiopurin-9-yl)-5-(hydroxymethyl)oxolane-3,4-diol (8)

¹H NMR (Me₂SO-d₆) δ 1.60 (m, 6H, cyclopentyl), 2.15 (m, 2H, cyclopentyl), 3.57 (m, 2H, CH₂-5′), 3.92 (m, 2H, H-4′ and CHS), 4.12 (m 1H, H-3′), 4.64 (m, 1H, H-2′), 5.82 (d, J=5.9 Hz, 1H, H-1′), 7.36 (bs, 2H, NH₂), 8.24 (s, 1H, H-8).

### Preparation of 2-(ar)alkylthioadenosine-5′-N-ethyluronamides (1a-5a)

A mixture of 2-iodoadenosine-5′-N-ethyluronamide (2-iodoNECA) (1, 0.15 g, 0.35 mmol prepared as described in Cristalli et al J. Med. Chem. 1994, Vol. 37, p. 1720-1726) in 5 mL of dry DMF, 1.75 mmol of the appropriate mercaptan, and solid K₂CO₃ (150 mg, 1.05 mmol) was heated in a steel bomb at 120° C. for the time reported in Table 1. The reaction mixture was concentrated in vacuo and the residue was chromatographed on a silica gel column eluting with the suitable mixture of solvents (Table 2) to give 1a-5a as chromatographically pure solids.

### [(2S, 3S, 4R, 5R)-5-(6-amino-2-phenylthiopurin-9-yl)-3,4-dihydroxyoxolan-2yl]-N -ethylcarboxamide (1a)

¹H NMR (Me₂SO-d₆) δ1.04 (t, J=7.1 Hz, 3H, CH₂CH₃), 3.18 (m, 2H, CH₂CH₃), 4.12 (m 1H, H-3′), 4.27 (s 1H, H-4′), 4.65 (m, 1H, H-2′), 5.82 (d, J=7.2 Hz, 1H, H-1′), 7.45 (m, 5H, H-Ph and NH₂), 7.58 (m, 2H, H-Ph), 8.14 (t, J=5.9 Hz, 1H, NH), 8.37 (s, 1H, H-8). Anal. (C₁₈H₂₀N₆O₄S).

### [(2S, 3S, 4R, 5R)-5-(6-amino-2-(phenylmethylthio)purin-9-yl)-3,4-dihydroxyoxolan-2yl]-N-ethylcarboxamide (2a)

¹H NMR (Me₂SO-d₆) δ1.02 (t, J=6.8 Hz, 3H, CH₂CH₃), 3.19(m, 2H, CH₂CH₃), 4.18 (m, 1H, H-3′), 4.31 (s 1H, H-4′), 4.38 (s, 2H, CH₂-S), 4.64 (m, 1H, H-2′), 5.96 (d, J=6.7 Hz, 1H, H-1′), 7.26 (m, 3H, H-Ph), 7.50 (m, 4H, H-Ph and NH₂), 8.27 (t, J=5.4 Hz, 1H, NH), 8.36 (s, 1H, H-8). Anal. (C₁₉H₂₂N₆O₄S).

### [(2S, 3S, 4R, 5R)-5-(6-amino-2-(phenylethylthio)purin-9-yl)-3,4-dihydroxyoxolan-2-yl]-N-ethylcarboxamide (3a)

¹H NMR (Me₂SO-d₆) δ 1.03 (t, J=7.0 Hz, 3H, CH₂CH₃), 2.97 (m, 2H, CH₂Ph), 3.10-3.50 (m, 4H, CH₂CH₃ and CH₂S), 4.19 (m, 1H, H-3′), 4.32 (s, 1H, H-4′), 4.70 (m, 1H, H-2′), 5.98 (d, J=7.0 Hz, 1H, H-1′), 7.39 (m, 5H, H-Ph), 7.50 (bs,2H, NH₂), 8.26 (t, J=5.8 Hz, 1H, NH), 8.36 (s, 1H, H-8).

### [(2S, 3S, 4R, 5R)-5-(6-amino-2-(phenylpropylthio)purin-9-yl)-3,4-dihydroxyoxolan-2-yl]-N-ethylcarboxamide (4a)

¹H NMR (Me₂SO-d₆) δ 1.05 (t, J=7.1 Hz, 3H, CH₂CH₃), 1.97 (m, 2H, CH₂CH₂Ph), 273 (m, 2H, CH₂Ph), 2.97-3.34 (m, 4H, CH₂CH₃ and CH₂S), 4.23 (m, 1H, H-3′), 4.32 (s, 1H, H-4′), 4.62 (m, 1H, H-2′), 5.94 (d, J=7.2 Hz, 1H, H-1′), 7.28 (m, 5H, H-Ph), 7.45 (bs, 2H NH₂), 8.25 (t, J=5.7 Hz, 1H, NH), 8.34 (s, 1H, H-8).

### [(2S, 3S, 4R, 5R)-5-(6-amino-2-pentylthiopurin-9-yl)-3,4-dihydroxyoxolan-2-yl]-N-ethylcarboxamide (5a)

¹H NMR (Me₂SO-d₆) δ 0.89 (t, J=XX Hz, (CH₂)₄CH₃), 1.04 (t, J=7.0 Hz, CH₂CH₃), 1.32 (m, 4H, (CH₂)₂CH₃), 1.67 (m, 2H, CH₂CH₂S), 3.12 (m, 4H, CH₂CH₃ and CH₂S), 4.20 (m, 1H, H-3′), 4.30 (s, 1H, H-4′), 4.72 (m, 1H, H-2′), 5.92 (d, J=7.2 Hz, 1H, H-1′), 7.45 (bs, 2H, NH₂), 8.24 (t, J=5.9 Hz, 1H, NH), 8.33 (s, 1H, H-8).

### EXAMPLE 3

Compositions of this invention were assayed to determine their affinity for the A_{2A} receptor in a pig striatum membrane prep. 0.2 mg of pig striatal membranes were treated with adenosine deaminase and 50 mM Tris buffer (pH=7.4) followed by mixing. To the pig membranes was added 2 μL of serially diluted DMSO stock solution of the compounds of this invention at concentrations ranging from 100 μM to 10 nM or the control received 2 μL of DMSO alone, then the tritiated antagonist ZM 241385 in Tris buffer (50 mM, pH of 7.4) was added to achieve a final concentration of 2 nM . After incubation at 23° C. for 2h, the solutions were filtered using a membrane harvester using multiple washing of the membranes (3×). The filter disks were counted in scintillation cocktail affording the amount of displacement of tritiated ZM by the competitive binding compositions of this invention. Greater than a 5 point curve was used to generate IC50's and the number of experiments (n) is indicated in the column marked in Tables 3-4 below. The Chang-Prusoff equation was used to generate Ki's from the IC₅₀ data.

Table 3. The affinity of novel A2A compounds for the A2A adenosine receptor of Pig striatal membranes.

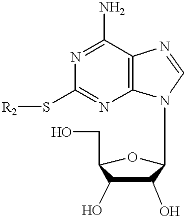

| | | A_{2A} |
|---|---|---|
| Cp. | R₂ | (Ki; nM) |
| 1 | Ph— | >10,000 |
| 2 | Ph—CH₂— | 9,537 |
| 3 | Ph—(CH₂)₂— | 892 |
| 4 | Ph—(CH₂)₃— | >10,000 |
| 5 | CH₃—(CH₂)₄— | >10,000 |
| 6 | HO—(CH₂)₄— | >100,000 |
| 7 | cyclopentyl- | >10,000 |

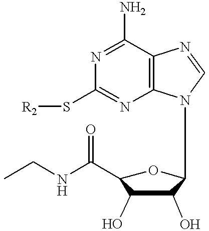

### 1 a-5a

| | | A_{2A} | A₁ |
|---|---|---|---|
| Cp. | R₂ | (Ki; nM) | (Ki; nM) |
| 1a | Ph— | 6,123 | |
| 2a | Ph—CH₂— | >10,000 | |
| 3a | Ph—(CH₂)₂— | 85 | >10,000 |
| 4a | Ph—(CH₂)₃— | 6,279 | |
| 5a | CH₃—(CH₂)₄— | 4,306 | |

These results indicate that the compositions of this invention are potent enough to be useful as vasodilators.

What we claim is:

1. A composition of matter having the following formula: 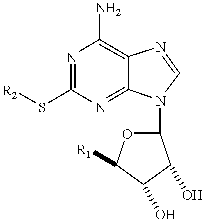
R¹ is —CH₂OH or —C(═O)NR⁷R⁸;
R² is selected from the group consisting of C₁₋₁₅ alkyl, aryl, and heteroaryl, which alkyl, aryl, and heteroaryl are optionally substituted with from 1 to 2 substituents independently selected from the group consisting of halo, NO₂, heterocyclyl, aryl, heteroaryl, CF₃, CN, OR^{°}, SR²⁰, N(R²⁰)₂, S(O)R²², SO₂ R²²SO²N(R²⁰)₂, SO₂NR²⁰COR²², SO₂NR²⁰CO₂R²², SO₂NR²⁰CON(R²⁰)₂, NR²⁰COR²², NR²⁰CO₂R²²NR²⁰ CON(R²⁰)₂, NR²⁰C(NR²⁰)NHR²³, COR²⁰, CO₂R²⁰, CON(R²⁰)₂, and wherein each optional heteroaryl, aryl, and heterocyclyl substitution substituent is further optionally substituted with halo, alkyl, CF₃, amino, mono- or di-alkylamino, SR²⁰, S(O)R²², SO₂R²², SO₂N(R²⁰)₂, CN, or OR²⁰;
R⁷ and R⁸ are each independently selected from H, and C₁₋₆ alkyl optionally substituted with 1 substituent independently selected from the group consisting of aryl and heteroaryl;
R²⁰ is selected from the group consisting of H, C₁₋₆ alkyl, aryl, and heteroaryl, which alkyl, aryl, and heteroaryl are each optionally substituted with from 1 to 2 substituents independently selected from halo, alkyl, mono- or dialkylamino, alkyl or aryl or heteroaryl amide, CN, O—C-₁₋₆ alkyl, CF₃; and
R²² is selected from the group consisting of C₁₋₆ alkyl, aryl, and heteroaryl which alkyl, aryl, and heteroaryl are each optionally substituted with from 1 to 2 substituents independently selected from halo, alkyl, mono- or dialkylamino, CN, —O—C₁₋₆ alkyl, CF₃.

2. The composition of claim 1 wherein R¹ is —CH₂OH or —C(═O)NR⁷R⁸;
R² is selected from the group consisting of C₁₋₉ alkyl, aryl, and heteroaryl, which alkyl, aryl, and heteroaryl are optionally substituted with from 1 to 2 substituents independently selected from the group consisting of halo, NO₂, aryl, CF₃, CN, OR²⁰, SR²⁰, N(R²⁰)₂, COR²⁰, CO₂R²⁰, CON(R²⁰)₂, and wherein each optional aryl substituent is further optionally substituted with halo, alkyl, CF₃, CN, or OR²⁰;
R⁷ and R⁸ are each independently selected from H, and C₁₋₂ alkyl; and
R²⁰ is selected from the group consisting of H, C₁₋₃ alkyl.

3. The composition of claim 1 wherein R¹ is —CH₂)H or —C(═O)NR⁷R⁸;
R² is selected from the group consisting of C₁₋₆ alkyl and aryl which alkyl and aryl are optionally substituted with from 1 to 2 substituents independently selected from the group consisting of halo, aryl, CF₃, CN, OR²⁰, and wherein each optional aryl substituent is further optionally substituted with halo, alkyl, CF₃, CN, or OR²⁰;
R⁷ and R⁸ are each independently selected from H, and C₁₋₂ alkyl; and
R²⁰ is selected from the group consisting of H and methyl.

4. The composition of claim 1 wherein R¹is —CH₂H or —C(═O)NR⁷R⁸;
R² is selected from the group consisting of C₁₋₅ alkyl and aryl which alkyl and aryl are optionally substituted with 1 substituent independently selected from the group consisting of halo, aryl, CF₃, CN, OR²⁰, and wherein each optional aryl substituent is further optionally substituted with halo, alkyl, CF₃;
R⁷ and R⁸ are each independently selected from H, and C₁₋₂ alkyl; and
R²⁰ is selected from the group consisting of H and methyl.

5. The composition of claim 1, or 2, or 3 wherein R¹ is —C(═O)NR⁷R⁸.

6. The composition of 4 wherein R¹ is —C(═O)NR⁷R⁸.

7. The composition of claim 6 wherein R² is C₁₋₅ alkyl which alkyl is optionally substituted with 1 substituent independently selected from the group consisting of aryl, CF₃, CN, OR²⁰, and wherein each optional aryl substituent is further optionally substituted with halo, alkyl, CF₃;
R⁷ and R⁸ are each independently selected from H, and C₁₋₂ alkyl; and
R²⁰ is selected from the group consisting of H and methyl.

8. The composition of claim 6 wherein R² is C₁₋₅ alkyl which alkyl is optionally substituted with 1 substituent independently selected from the group consisting of aryl, OR²⁰, and wherein each optional aryl substituent is further optionally substituted with halo;
R⁷ and R⁸ are each independently selected from H, and C₁₋₂ alkyl; and
R²⁰ is selected from the group consisting of H and methyl.

9. The composition of claim 1 wherein R¹ is —C(═O)NHEt; wherein R² is C ₁₋₅ alkyl which alkyl is optionally substituted with 1 substituent independently selected from the group consisting of aryl, OR²⁰; and R²⁰ is H.

10. The composition of claim 6 wherein R² is aryl, which aryl is optionally substituted with 1 substituent independently selected from the group consisting of CF₃, CN, OR²⁰; R⁷ and R⁸ are each independently selected from H, and C₁₋₂ alkyl; and R²⁰ is selected from the group consisting of H and methyl.

11. The composition of claim 1 wherein R¹ is —C(═O)NHEt; R² is aryl, which aryl is optionally substituted with 1 substituent independently selected from the group consisting of CF₃, CN, OR²⁰; and R²⁰ is selected from the group consisting of H and methyl.

12. The composition of claim 1, or 2, or 3 wherein R¹ is —CH₂OH.

13. The composition of 4 wherein R¹ is —CH₂OH.

14. The composition of claim 13 wherein R² is C₁₋₅ alkyl which alkyl is optionally substituted with 1 substituent independently selected from the group consisting of aryl, CF₃, CN, OR²⁰, and wherein each optional aryl substituent is further optionally substituted with halo, alkyl, CF₃;
R⁷ and R⁸ are each independently selected from H, and C₁₋₂ alkyl; and
R²⁰ is selected from the group consisting of H and methyl.

15. The composition of claim 13 wherein R² is C₁₋₅ alkyl which alkyl is optionally substituted with 1 substituent independently selected from the group consisting of aryl, OR²⁰, and wherein each optional aryl substituent is further optionally substituted with halo;
R⁷ and R⁸ are each independently selected from H, and C₁₋₂ alkyl; and
R²⁰ is selected from the group consisting of H and methyl.

16. The composition of claim 13 wherein R² is aryl which aryl is optionally substituted with 1 substituent independently selected from the group consisting of CF₃, CN, OR²⁰; R⁷ and R⁸ are each independently selected from H, and C₁₋₂ alkyl; and R²⁰ is selected from the group consisting of H and methyl.

17. The composition matter of claim 1 wherein the compound is selected from the group consisting (4S, 2R, 3R, 5R)-2-(6-amino-2-phenylthiopurin-9-yl)-5-(hydroxymethyl)oxolane-3,4-diol, (4S, 2R, 3R, 5R)-2-(6-amino-2-phenylmethylthiopurin-9-yl)-5-(hydroxymethyl)oxolane-3,4-diol, (4S, 2R, 3R, 5R)-2-(6-amino-2-phenylethylthiopurin -9-yl)-5-(hydroxymethyl)oxolane-3,4-diol, (4S, 2R, 3R, 5R)-2-(6-amino-2-phenylpropylthiopurin-9-yl)-5-(hydroxymethyl)oxolane-3,4-diol, (4S, 2R, 3R, 5R)-2-(6-amino-2-pentylthiopurin-9-yl)-5-(hydroxymethyl)oxolane-3,4-diol, (4S, 2R, 3R, 5R)-2-(6-amino-2-(4-hydroxybutylthio)purin-9-yl)-5-(hydroxymethyl)oxolane-3,4-diol, (4S, 2R, 3R, 5R)-2-(6-amino-2-cyclopentylthiopurin-9-yl)-5-(hydroxymethyl)oxolane-3,4-diol, [(2S, 3S, 4R, 5R)-5-(6-amino-2-phenylthiopurin-9-yl)-3,4-dihydroxyoxolan-2yl]-N-ethylcarboxamide, [(2S, 3S, 4R, 5R)-5-(6-amino-2-(phenylmethylthio)purin-9-yl)-3,4-dihydroxyoxolan-2yl]-N -ethylcarboxamide, [(2S, 3S, 4R, 5R)-5-(6-amino-2-(phenylethylthio)purin-9-yl)-3,4-dihydroxyoxolan-2-yl]-N-ethylcarboxamide, [(2S, 3S, 4R, 5R)-5-(6-amino-2-(phenylpropylthio)purin-9-yl)-3,4-dihydroxyoxolan-2-yl]-N-ethylcarboxamide, [(2S, 3S, 4R, 5R)-5-(6-amino-2-pentylthiopurin-9-yl)-3 ,4-dihydroxyoxolan-2-yl]-N-ethylcarboxamide,a and mixtures thereof.

18. A method for stimulating coronary vasodilatation in a mammal by administering to the mammal a therapeutically effective amount of a compound of claim 1 that is sufficient to stress the heart and induce a coronary steal situation for the purposes of imaging the heart.

19. The method of claim 18 wherein the therapeutically effective amount ranges from about 0.01 to about 100 mg/kg weight of the mammal.

20. The method of claim 18 wherein the mammal is a human.

21. A pharmaceutical composition of matter comprising the composition of claim 1 and one or more pharmaceutical excipients.

22. The pharmaceutical composition of matter of claim 21 wherein the pharmaceutical composition is in the form of a solution.

23. The pharmaceutical composition of matter of claim 21 wherein the composition is useful as an anti-inflammatory, in adjunctive therapy with angioplasty, as a platelet aggregation inhibitor, and as an inhibitor of platelet and neutrophil activation.
